(19) United States
(12) Patent Application Publication
(10) Pub. No.: US 2013/0137082 A1
(43) Pub. Date: May 30, 2013

(54) BIOSENSOR, APPARATUS AND METHOD FOR DETECTING A BIOMOLECULE USING THE BIOSENSOR

(71) Applicants: Chan Woo PARK, Daejeon (KR); Jong-Heon YANG, Daejeon (KR); Chil Seong AH, Daejeon (KR); Wan Joong KIM, Goyang (KR); Kwang Hyo CHUNG, Daejeon (KR); Yo Han CHOI, Daejeon (KR); Gun Yong SUNG, Daejeon (KR)

(72) Inventors: Chan Woo PARK, Daejeon (KR); Jong-Heon YANG, Daejeon (KR); Chil Seong AH, Daejeon (KR); Wan Joong KIM, Goyang (KR); Kwang Hyo CHUNG, Daejeon (KR); Yo Han CHOI, Daejeon (KR); Gun Yong SUNG, Daejeon (KR)

(73) Assignee: Electronics and Telecommunications Research Institute, Daejeon (KR)

(21) Appl. No.: 13/545,894

(22) Filed: Jul. 10, 2012

(30) Foreign Application Priority Data
Nov. 24, 2011 (KR) 10-2011-0123607

(51) Int. Cl. C12M 1/34; G01N 27/26; B82Y 15/00; B82Y 5/00

(52) U.S. Cl. 435/5; 435/6.19; 435/6.11; 435/7.1; 435/7.4; 435/287.2; 435/287.1; 436/501; 422/69; 977/774; 977/700; 977/920

(57) ABSTRACT

Provided are a biosensor, an apparatus and a method for detecting a biomolecule using the biosensor. The biosensor may include a supporting substrate, a semiconductor layer spaced apart from a top surface of the supporting substrate by supporting patterns, and a nano-motor array formed on a top surface of the semiconductor layer. The nano-motor array may include a plurality of nano-metal rods configured to exhibit an autonomous propulsion in a fluid.

### CROSS-REFERENCE TO RELATED APPLICATIONS

This U.S. non-provisional patent application claims priority under 35 U.S.C. §119 to Korean Patent Application No. 10-2011-0123607, filed on Nov. 24, 2011, in the Korean Intellectual Property Office, the entire contents of which are hereby incorporated by reference.

### BACKGROUND OF THE INVENTION

Embodiments of the inventive concepts relate to a biosensor, an apparatus and method for detecting a biomolecule using the biosensor, and in particular, a biosensor with a nano-motor array, an apparatus and a method for detecting a biomolecule using the biosensor.

Metal nanorods including a hetero junction type metal pair (e.g., a pair of platinum (Pt) and gold (Au) or a pair of nickel (Ni) and gold (Au)) may exhibit an autonomous propulsion in a hydrogen peroxide solution. Such a nanostructure, exhibiting the autonomous propulsion, is known as a “nano-motor”, which can be used for several technical fields, such as high-efficient drug carriers, diagnosis of diseases of human, and mobile sensors for treating a disease.

Recently, it was reported that the presence of silver ions (Ag+) provided in the hydrogen peroxide solution can greatly increase the autonomous propulsion property of the Pt—Au nano-motor. Based on the phenomena, a biosensor has been developed to measure a concentration of target biomolecules labeled with silver nanoparticles. The biosensor may include Pt—Au nano-motors formed on a mold, which may be formed of anodized aluminum oxide (AAO) with nano-sized pores, in a bottom-up manner by using an electroplating process, and it may be configured to trace movements of the Pt—Au nano-motors distributed in a sample solution by using an optical microscope. In more detail, a reacting part may be provided to capture the target biomolecules labeled with silver nanoparticles in the sample solution. Here, the higher a concentration of the target molecule in the sample solution be, the more the silver nanoparticles are bound with the reacting part. In other words, in the case where the target molecule has an increased concentration, an amount of silver ions dissolved from the silver nanoparticle increases and consequently the nano-motor has an increased mobility, even in the condition of the same hydrogen peroxide concentration. Accordingly, an amount of the target molecules in a solution can be quantitatively estimated by measuring a moving speed (or a moving distance per unit time) of the nano-motor.

However, in this method, since the nano-motors in the solution are in random or Brownian motion rather than linear motion, it is hard to measure the moving speed of each nano-motor. Alternatively, a thin nickel layer having magnetism may be provided in the gold layer, and an external magnetic field may be used to realize linear movements of the nano-motors.

However, this modified method suffers from low levels of reproducibility and reliability as well as a long analysis time, because only movements of selected ones of the nano-motors can be traced by a visual monitoring method. Furthermore, in this modified method, additional equipment, such as an optical microscope or a camera, should be provided to trace the movements of the nano-motors. Accordingly, it is hard to use this modified method to realize a portable or real-time analysis device suitable for onsite diagnoses.

### SUMMARY

Embodiments of the inventive concepts provide a biosensor, which can be fabricated in a small size and have a real-time detection function, and an apparatus and a method for detecting a biomolecule using the biosensor.

According to example embodiments of the inventive concepts, a biosensor may include a supporting substrate, a semiconductor layer spaced apart from a top surface of the supporting substrate by supporting patterns, and a nano-motor array formed on a top surface of the semiconductor layer, the nano-motor array including a plurality of nano-metal rods configured to exhibit an autonomous propulsion in a fluid.

In example embodiments, the nano-metal rod may have a structure, in which different metal layers may be jointed with each other.

In example embodiments, the nano-metal rod may be provided as a form of a Pt—Au metal pair or a Ni—Au metal pair.

In example embodiments, the nano-metal rod may be configured to be moved along a specific direction in a hydrogen peroxide solution, thereby exhibiting the autonomous propulsion.

In example embodiments, the nano-metal rod may be configured to apply a stress to the semiconductor layer, when the nano-motor array may be provided in the fluid.

In example embodiments, the semiconductor layer may be formed of a semiconductor material, whose electric conductivity varies substantially depending on the autonomous propulsion from the nano-metal rods.

In example embodiments, the semiconductor layer has a thickness ranging from 1 nm to 100 nm.

According to example embodiments of the inventive concepts, an apparatus for detecting a biomaterial may include a biomaterial reacting part immobilized with probe molecules, the probe molecules being specifically bound with target molecules labeled with silver nanoparticles, a biomaterial detecting part provided with a nano-motor array including a plurality of nano-metal rods, the nano-metal rods exhibiting an autonomous propulsion in a fluid, and a fluid channel supplying the fluid to the biomaterial reacting part and the biomaterial detecting part.

In example embodiments, the biomaterial detecting part may be configured to detect a change in electric conductivity of the semiconductor layer between before and after providing the nano-motor array in the fluid.

In example embodiments, in the biomaterial detecting part, the autonomous propulsion of the nano-metal rods may be dependent on a concentration of silver ions dissolved in the fluid.

In example embodiments, the biomaterial detecting part may further include a supporting substrate, a semiconductor layer provided on a top surface of the supporting substrate by supporting patterns interposed between the supporting substrate and the semiconductor layer to separate the supporting substrate from the semiconductor layer, and the nano-metal rods may be provided on a top surface of the semiconductor layer.

In example embodiments, the nano-metal rod may be configured to apply a stress to the semiconductor layer, when the nano-motor array of the biomaterial detecting part may be provided in the fluid.

In example embodiments, the semiconductor layer may be formed of a semiconductor material, whose electric conductivity varies substantially depending on the autonomous propulsion from the nano-metal rods.

In example embodiments, each of the nano-metal rods may be provided as a form of a Pt—Au metal pair or a Ni—Au metal pair configured to be moved along a specific direction in a hydrogen peroxide solution.

According to example embodiments of the inventive concepts, a method of detecting a biomaterial may be performed by using a biomaterial-detecting apparatus with a biomaterial reacting part and a biomaterial detecting part. The biomaterial detecting part may be formed on a semiconductor layer and be provided with a nano-motor array including a plurality of nano-metal rods exhibiting an autonomous propulsion in a fluid. Here, the method may include measuring a first electric conductivity of the semiconductor layer, immobilizing target molecules labeled with silver nanoparticles on the biomaterial reacting part, supplying a fluid to the biomaterial reacting part immobilized with the target molecules and the biomaterial detecting part, and measuring a second electric conductivity of the semiconductor layer, during the supplying of the fluid.

In example embodiments, the method may further include determining a concentration of the target molecule using a difference between the first electric conductivity and the second electric conductivity.

In example embodiments, the determining of a concentration of the target molecule may be performed by using a change in a concentration of silver ion caused by dissolution of the silver nanoparticle by the fluid.

In example embodiments, the nano-metal rods may be provided as a form of a Pt—Au metal pair or a Ni—Au metal pair, and the supplying of the fluid may include supplying a hydrogen peroxide solution.

In example embodiments, the supplying of the fluid may include supplying a hydrogen peroxide solution.

In example embodiments, the immobilizing of the target molecules labeled with silver nanoparticles may include providing a substrate immobilized with first probe molecules, which may be specifically bound with the target molecules, to the biomaterial reacting part, providing the target molecules to the biomaterial reacting part to bind the target molecules specifically with the first probe molecules, and providing second probe molecules immobilized on a surface of the silver nanoparticle to the biomaterial reacting part to bind the second probe molecules specifically with the target molecules.

### BRIEF DESCRIPTION OF THE DRAWINGS

Example embodiments will be more clearly understood from the following brief description taken in conjunction with the accompanying drawings. The accompanying drawings represent non-limiting, example embodiments as described herein.

FIG. 1 is a schematic diagram illustrating a biosensor according to example embodiments of the inventive concept.

FIGS. 2A through 2D are diagrams illustrating a reacting part of a biosensor according to example embodiments of the inventive concept.

FIG. 3 is a schematic perspective view illustrating a detecting part of a biosensor according to example embodiments of the inventive concept.

FIG. 4 is a schematic diagram illustrating an operational principle of a nano-motor constituting a detecting part of a biosensor according to example embodiments of the inventive concept.

FIG. 5 is a flow chart illustrating a method of detecting a biomaterial using a biosensor according to example embodiments of the inventive concept.

FIGS. 6A and 6B are diagrams illustrating a method of detecting a biomaterial using a biosensor according to example embodiments of the inventive concept.

FIGS. 7A through 7D are diagrams illustrating a method of fabricating a biosensor according to example embodiments of the inventive concept.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION

Example embodiments of the inventive concepts will now be described more fully with reference to the accompanying drawings, in which example embodiments are shown. Example embodiments of the inventive concepts may, however, be embodied in many different forms and should not be construed as being limited to the embodiments set forth herein; rather, these embodiments are provided so that this disclosure will be thorough and complete, and will fully convey the concept of example embodiments to those of ordinary skill in the art. In the drawings, the thicknesses of layers and regions are exaggerated for clarity. Like reference numerals in the drawings denote like elements, and thus their description will be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items. Other words used to describe the relationship between elements or layers should be interpreted in a like fashion (e.g., “between” versus “directly between,” “adjacent” versus “directly adjacent,” “on” versus “directly on”).

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Example embodiments of the inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the inventive concepts should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

In the present specification, the term “target molecule” means a biomolecule to be analyzed and may have the same technical meaning as clinical specimen or analyte.

In the present specification, the term “probe molecule” means a biomolecule to be specifically bound with the target molecule and may have the same technical meaning as a detecting material, a receptor, or an acceptor.

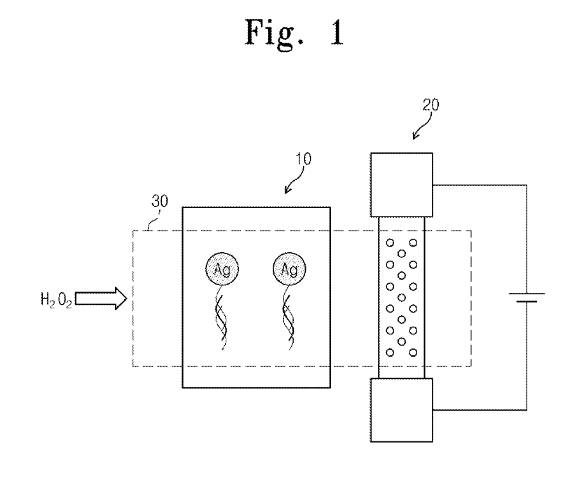

FIG. 1 is a schematic diagram illustrating a biosensor according to example embodiments of the inventive concept.

Referring to FIG. 1, a biosensor may include a biomaterial reacting part 10, in which reactions of, or between, biomaterials may occur, and a biomaterial detecting part 20 detecting a specific biomaterial. In addition, the biosensor may further include a fluid channel 30, which may supply a fluid to the biomaterial reacting part 10 and the biomaterial detecting part 20.

In example embodiments, target molecules labeled with silver nanoparticles may be provided in the biomaterial reacting part 10. For example, the biomaterial reacting part 10 may include a substrate immobilized with first probe molecules, which may be specifically bound with the target molecules. On the substrate, the target molecule may be specifically bound with first and second probe molecules by a sandwich immune response. The second probe molecules may be labeled with the silver nanoparticles. In other words, a conjugate consisting of the first probe molecule, the target molecule, the second probe molecule, and the silver nanoparticle may be immobilized on the substrate of the biomaterial reacting part 10.

The biomaterial detecting part 20 may include a semiconductor layer and a nano-motor array on the semiconductor layer. The biomaterial detecting part 20 may be configured to detect a concentration of the target molecule in the biomaterial reacting part 10. For example, the nano-motor array may be configured to exhibit autonomous propulsion depending on a concentration of a target molecule, and the concentration of the target molecule may be detected by using a change in the autonomous propulsion of the nano-motor array.

The fluid channel 30 may supply solutions, which may be used to detect the biomaterial, to the biomaterial reacting part 10 and the biomaterial detecting part 20. In example embodiments, a solution containing the target molecules and a hydrogen peroxide (H₂O₂) solution may be supplied into the biomaterial reacting part 10 and the biomaterial detecting part 20 via the fluid channel 30.

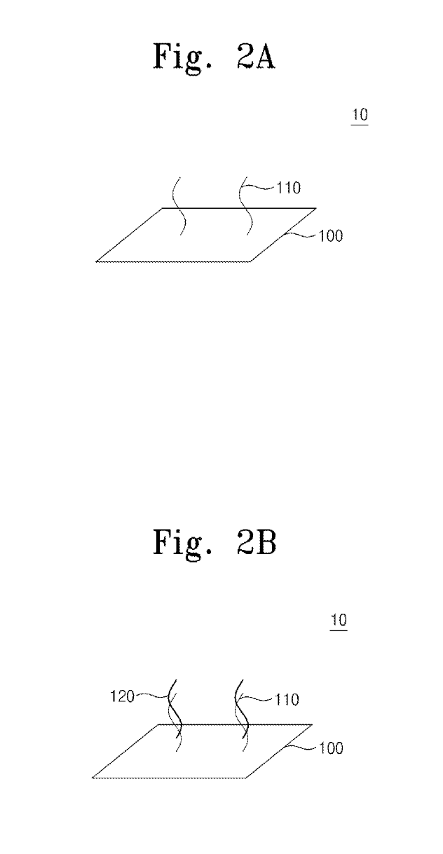

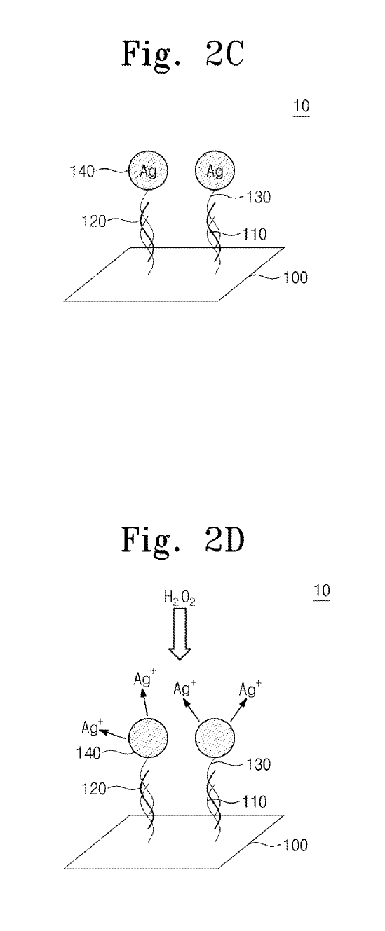

FIGS. 2A through 2D are diagrams illustrating a reacting part of a biosensor according to example embodiments of the inventive concept.

Referring to FIG. 2A, first probe molecules 110 may be immobilized on a surface of a substrate 100 provided in the biomaterial reacting part 10.

In example embodiments, the substrate 100 may be formed to define a fluid channel, and be formed of, for example, plastic, glass, or semiconductor. To facilitate the immobilization of the first probe molecules 110, the substrate 100 may be prepared to have improved surface properties, such as, good surface orientation, uniform spatial distribution, and easily functionalizable structure.

The first probe molecules 110 may be bio molecules having binding sites that can be specifically bound with the target molecule. The first probe molecules 110 may include, for example, protein, cell, virus, nucleic acid, organic molecules, or inorganic molecules. In the case of protein, the first probe molecules 110 may be all biomaterials such as antigen, antibody, matrix protein, enzyme and coenzyme. In the case of nucleic acid, the first probe molecules 110 may be DNA, RNA, PNA, LNA or a hybrid thereof. In example embodiments, the first probe molecule 110 may be a monoclonal antibody or a polyclonal antibody.

The first probe molecules 110 may be immobilized on the surface of the substrate 100 through chemical adsorption, covalent-binding, electrostatic attraction, co-polymerization, or an avidin-biotin affinity system.

In addition, the first probe molecules 110 may be immobilized on the surface of the substrate 100 by using a linker. One of self-assembled monolayer (SAM), polyethylene glycol (PEG), dextran, or protein G may be used for the linker. Furthermore, a functional group, such as, a carboxyl group (—COOH), a thiol group (—SH), a hydroxyl group (—OH), a silane group, an amine group, or an epoxy group, may be induced on the surface of the substrate 100. In addition, casein may be immobilized on the surfaces of the substrate 100 to prevent a nonspecific binding of target molecules 120.

Referring to FIG. 2B, a fluid containing the target molecules 120 may be supplied onto to the substrate 100 immobilized with the first probe molecules 110 of the biomaterial reacting part 10. The target molecules 120 may include a biomaterial, which can be specifically bound with the first probe molecules 110 immobilized on the substrate 100, and the target molecules 120 may be immobilized on the substrate 100 through a specific binding with the first probe molecules 110. The target molecules 120 may include, for example, protein, cell, virus, nucleic acid, organic molecules, or inorganic molecules. In the case of protein, the target molecules 120 may be all biomaterials such as antigen, antibody, matrix protein, enzyme and coenzyme. In the case of nucleic acid, the target molecules 120 may be DNA, RNA, PNA, LNA or a hybrid thereof. In example embodiments, the target molecules 120 may be an antigen.

Thereafter, a buffer solution may be supplied to the substrate 100 to remove the target molecules 120 that are not bound with the first probe molecules 110.

Referring to FIG. 2C, second probe molecules 130 labeled with silver nanoparticles 140 may be provided in the biomaterial reacting part 10.

The second probe molecules 130 may be a biomaterial having binding sites that can be specifically bound with the target molecules 120. The binding sites of the target molecules 120 to the second probe molecules 130 may differ from that of the target molecules 120 to the first probe molecules 110. In example embodiments, the second probe molecules 130 may be a polyclonal antibody.

The second probe molecules 130 may be immobilized on a surface of the silver nanoparticle 140, by a chemical adsorption, a covalent binding, an electrostatic attraction, a co-polymerization or an avidin-biotin affinity system.

The second probe molecules 130 immobilized on the silver nanoparticle 140 may be specifically bound with the target molecules 120 immobilized on the substrate 100. For example, a conjugate consisting of the first probe molecule 110, the target molecule 120, the second probe molecule 130, and the silver nanoparticle 140 may be immobilized on the substrate 100.

Referring to FIG. 2D, a hydrogen peroxide (H₂O₂) solution may be supplied to the substrate 100 of the biomaterial reacting part 10. Accordingly, the silver nanoparticles 140 may be dissolved into silver ions (Ag⁺) by the hydrogen peroxide solution. A concentration of silver ion in the hydrogen peroxide solution may be dependent on a concentration of the target molecules 120.

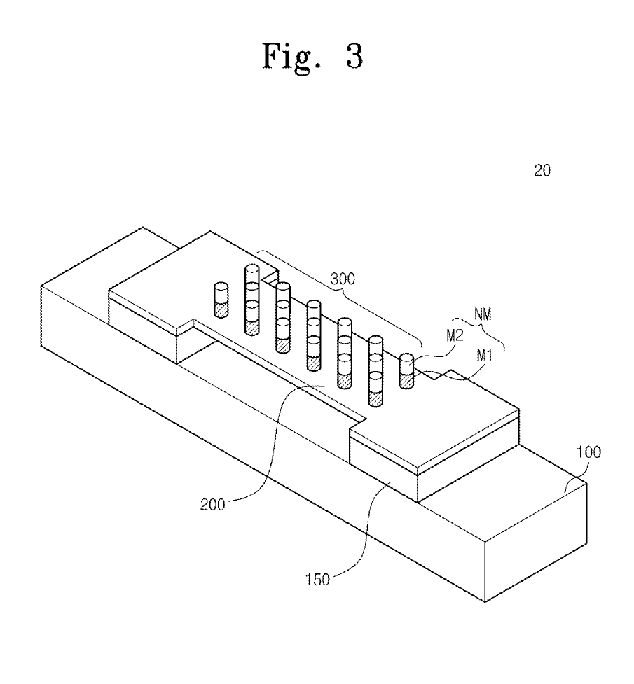

FIG. 3 is a schematic perspective view illustrating a detecting part of a biosensor according to example embodiments of the inventive concept.

Referring to FIG. 3, the biomaterial detecting part 20 may include the supporting substrate 100, a semiconductor layer 200, and a nano-motor array 300.

The supporting substrate 100 of the biomaterial detecting part 20 may be a part of a silicon-on-insulator (SOI) wafer or a part of one or more bulk semiconductor substrate. In example embodiments, the biomaterial detecting part 20 of the biosensor may be realized using the SOI wafer, and the supporting substrate 100 may include a layer formed of at least one of silicon (Si), germanium (Ge), silicon germanium (SiGe), oxides or carbon.

The semiconductor layer 200 may be vertically spaced apart from a top surface of the supporting substrate 100 by supporting patterns 150. For example, the supporting patterns 150 may be interposed between the semiconductor layer 200 and the supporting substrate 100 vertically spaced apart from each other. The supporting patterns 150 may be formed of an insulating material (for example, silicon oxide) and be disposed below both end portions of the semiconductor layer 200. In addition, electrodes (not shown) for measuring an electric current may be provided at the end portions of the semiconductor layer 200.

The semiconductor layer 200 spaced apart from the supporting substrate 100 may exhibit an electric resistance property depending on a stress applied thereto (i.e., a piezoresistive property). Electric conductivity of the semiconductor layer 200 may be measured through the electrodes provided at the end portions of the semiconductor layer 200.

In example embodiments, the semiconductor layer 200 may be an epitaxial layer formed on an insulating layer of the supporting substrate 100 using an epitaxial growth process. The semiconductor layer 200 may be formed to have a thickness of about 1-100 nm. In example embodiments, the supporting patterns 150 may be formed by selectively removing a portion of the insulating layer below the nano-motor array 300, after forming the semiconductor layer 200 and the nano-motor array 300.

The nano-motor array 300 may include a plurality of nano-metal rods NM provided on the semiconductor layer 200. The nano-metal rods NM may be configured to have an autonomous propulsion in a specific fluid. For example, each of the nano-metal rods NM may include two different metal layers jointed with each other (e.g., a bimetal structure). In example embodiments, each of the nano-metal rods NM may include a first metal M1 and a second metal M2 jointed with each other. For example, each of the nano-metal rods NM may be a pair of different metal layers (e.g., of platinum and gold or of nickel and gold). In example embodiments, each of the nano-metal rods NM may be shaped like a circular pillar or a cylinder with a diameter of several to several ten nanometers and with a height of several micrometers. For example, each nano-metal rod NM may be formed to have a diameter of about 100-200 nm and a height of about 1-2 μm.

The nano-metal rods NM may be disposed on the semiconductor layer 200 to be spaced apart from each other, and the first metal M1 (e.g., platinum (Pt) or nickel (Ni)) thereof may be in contact with the semiconductor layer 200. In example embodiments, the first metal M1 may serve as a decomposing the specific fluid. By contrast, the second metal M2 (e.g., gold (Au)) may be vertically spaced apart from the semiconductor layer 200.

In example embodiments, the nano-motor array 300 provided on the semiconductor layer 200 may apply a stress to the semiconductor layer 200, when it may be disposed in the hydrogen peroxide solution. Here, as the number of the nano-metal rods NM increases, the stress applied to the semiconductor layer 200 may increase. In addition, as a silver ion concentration in the hydrogen peroxide solution increases, the stress applied to the semiconductor layer 200 by the nano-metal rods NM may increase. The silver ion concentration may increase, as a concentration of the target molecule immobilized on the biomaterial reacting part increases. The increase of the silver ion concentration may lead to an increase in the autonomous propulsion of the nano-metal rods NM, thereby increasing the stress applied to the semiconductor layer 200.

In the case where a stress is applied to the semiconductor layer 200 spaced apart from the supporting substrate 100, the semiconductor layer 200 may be mechanically bent to have a changed electric resistance. Accordingly, it is possible to measure electric conductivity of the semiconductor layer 200, which may be changed by the stress applied to the semiconductor layer 200, using the biomaterial detecting part.

FIG. 4 is a schematic diagram illustrating an operational principle of a nano-motor constituting a detecting part of a biosensor according to example embodiments of the inventive concept.

Referring to FIG. 4, each nano-metal rod NM may include the first and second metals M1 and M2 jointed with each other. In example embodiments, the nano-metal rod NM may be a Pt—Au metal pair. If the Pt—Au metal pair is exposed by a hydrogen peroxide (H₂O₂) solution, platinum may serve as a catalyzer decomposing hydrogen peroxide into the water and oxygen and this may cause an autonomous propulsion, which means that the Pt—Au metal pair is moving toward the platinum layer. In other words, if the Pt—Au metal pair is exposed by a hydrogen peroxide containing material, hydrogen peroxide may be decomposed into the water and the oxygen on a surface of the nano-metal rod NM, and this lead to the autonomous propulsion moving the nano-metal rod NM toward the platinum layer.

In detail, when the hydrogen peroxide is decomposed on the surface of the platinum layer through a catalytic chemical reaction, an oxidation-reduction reaction of the hydrogen peroxide may occur at both sides of the nano-metal rod NM. The chemical reaction may produce an electron current in the nano-metal rod NM, and the nano-metal rod NM may have the autonomous propulsion.

Example embodiments of the inventive concept may not be limited to the embodiments, in which the nano-metal rod NM is the Pt—Au metal pair. In other words, the nano-metal rod NM may be realized using other metal pair selected to obtain the same technical effect as that of the Pt—Au metal pair.

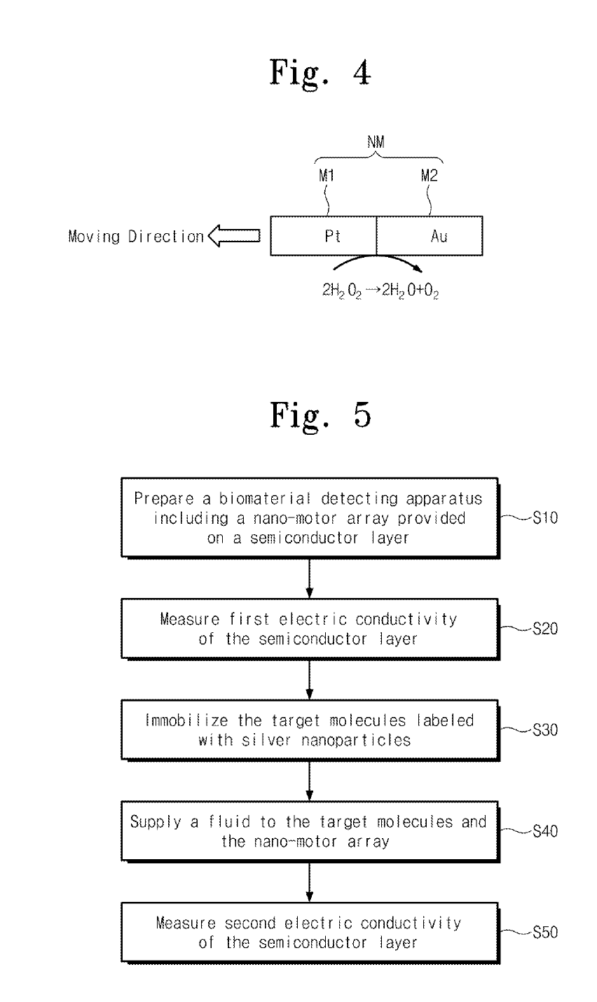

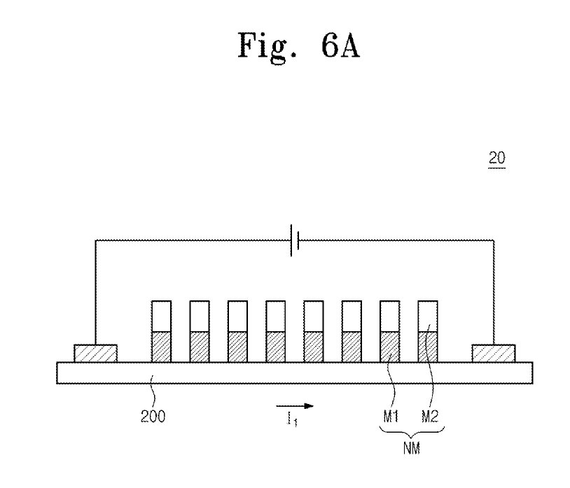

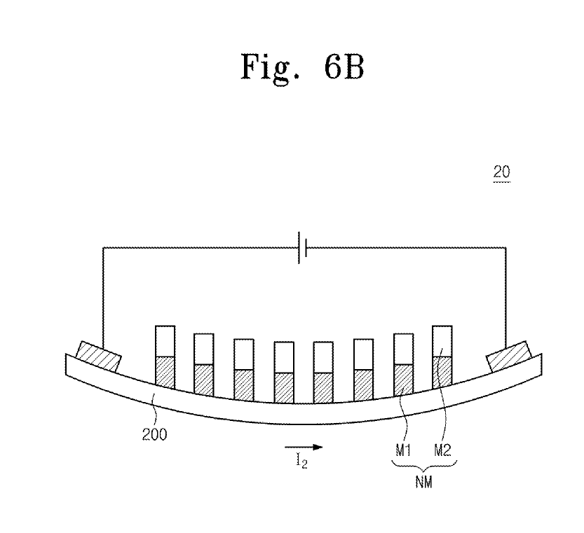

FIG. 5 is a flow chart illustrating a method of detecting a biomaterial using a biosensor according to example embodiments of the inventive concept. FIGS. 6A and 6B are diagrams illustrating a method of detecting a biomaterial using a biosensor according to example embodiments of the inventive concept.

Referring to FIG. 5, an apparatus for detecting a biomaterial may be prepared to include the nano-motor array provided on the semiconductor layer (in S10). For example, as shown in FIG. 1, the apparatus for detecting a biomaterial may be prepared to include the biomaterial reacting part 10 and the biomaterial detecting part 20. Here, the target molecules labeled with silver nanoparticles may not be proved in the biomaterial reacting part. For example, as shown in FIG. 2A, the substrate immobilized with the first probe molecules may be provided in the biomaterial reacting part.

Thereafter, in the absence of the target molecules labeled with silver nanoparticles, a first electric conductivity of the semiconductor layer may be measured by the biomaterial detecting part (in S20). For example, as shown in FIG. 6A, the first electric conductivity of the semiconductor layer 200 may be measured in the absence of the target molecules labeled with silver nanoparticles. In this case, the stress applied to the semiconductor layer 200 by the nano-metal rods NM may be substantially zero. In example embodiments, the measurement of the electric conductivity may include measuring an electric current I₁ between two electrodes provided on the semiconductor layer 200.

Next, the target molecules labeled with silver nanoparticles may be immobilized on the biomaterial reacting part by using the process described with reference to FIGS. 2B and 2C (in S30).

Thereafter, a predetermined fluid may be supplied to the target molecules labeled with silver nanoparticles and the nano-motor array 300 (in S40). For example, a hydrogen peroxide solution may be supplied to the biomaterial reacting part and the biomaterial detecting part. Accordingly, as shown in FIG. 2D, silver ions may be dissolved in the hydrogen peroxide solution, and a silver ion concentration in the hydrogen peroxide solution may vary depending on a concentration of the target molecule. Due to the presence of the hydrogen peroxide solution contained with the silver ions, the nano-metal rods NM of the biomaterial detecting part may produce an autonomous propulsion. For example, as shown in FIG. 6B, the semiconductor layer 200 may be deformed by the resultant force of autonomous propulsions applied from the nano-metal rods NM. Here, in the case where the first metal M1 serving as catalyst is a metal being in contact with the semiconductor layer 200, a stress from each nano-metal rod NM may be exerted from the semiconductor layer 200 toward the supporting substrate 100 (shown in FIG. 3). By contrast, in the case where the second metal M2, which may not be used as the catalyst, is a metal being in contact with the semiconductor layer 200, a stress from each nano-metal rod NM may be exerted from the supporting substrate 100 (in FIG. 3) toward the semiconductor layer 200.

The deformation of the semiconductor layer 200 caused by the stress or the autonomous propulsion from the nano-metal rod NM may lead to a change in electric conductivity of the semiconductor layer 200. In example embodiments, the biomaterial detecting part may be used to measure a second electric conductivity of the semiconductor layer 200 or an electric current I₂ between two electrodes (in S50).

In example embodiments, a difference between the first and second electric conductivities (or between I₁ and I₂) may be proportional to a concentration of the target molecule, and thus, the concentration of the target molecule can be examined by analyzing a change in electric conductivity of the semiconductor layer 200. In other words, it is possible to monitor a change in a silver ion concentration and/or a concentration of target molecule in real time.

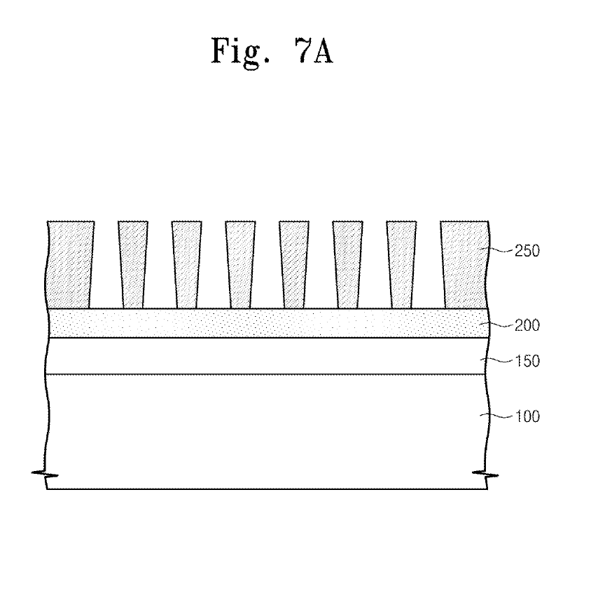

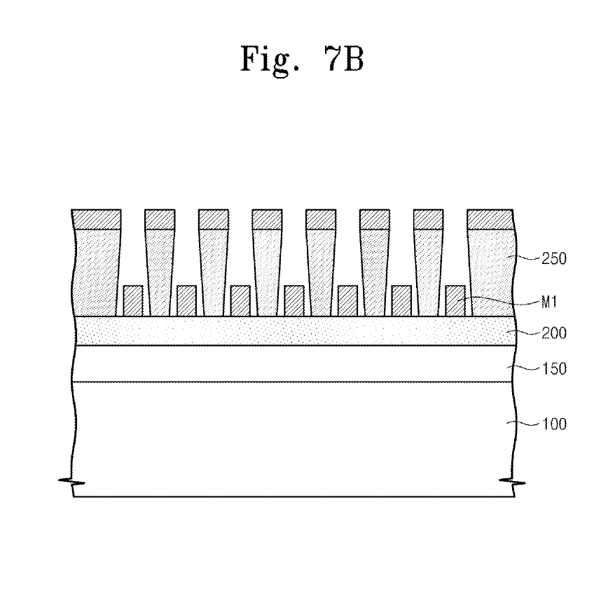

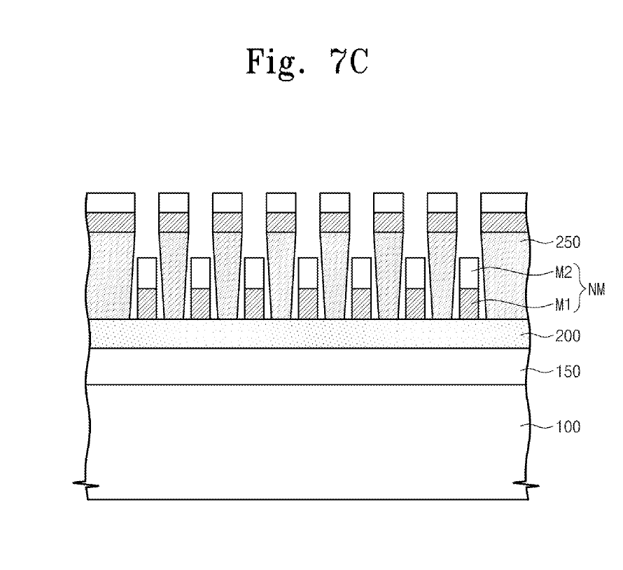

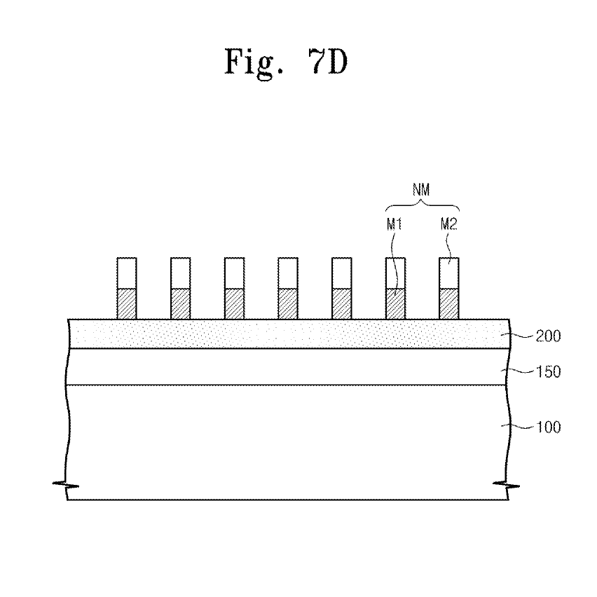

FIGS. 7A through 7D are diagrams illustrating a method of fabricating a biosensor according to example embodiments of the inventive concept.

In example embodiments, a nano-motor array may be formed by a top down process including a photolithography step, a metal deposition step, and a lift-off step. Accordingly, the nano-motor array may be formed to have a desired configuration at a desired position.

In detail, referring to FIG. 7A, a supporting structure including the supporting substrate 100, the buried oxide 150, and the semiconductor layer 200 may be prepared. In example embodiments, the supporting structure may be a wafer including an insulating layer and a single crystalline silicon layer thereon (e.g., a silicon-on-insulator (SOI) wafer).

A photoresist pattern 250 may be formed on the semiconductor layer 200. The photoresist pattern 250 may be formed to have openings, each of which has a width ranging from several nanometers to several ten nanometers. For example, the opening of the photoresist pattern 250 may have a width ranging from about 100 nm to about 200 nm. The photoresist pattern 250 may be formed on the semiconductor layer 200 using a photolithography process.

The photolithography process may include coating a photoresist film on a substrate, selectively exposing the photoresist film, and developing the exposed photoresist film.

Referring to FIG. 7B, the first metal layer M1 may be formed on the semiconductor layer 200 provided with the photoresist pattern 250. The first metal layer M1 may be formed on the semiconductor layer 200 exposed by the openings of the photoresist pattern 250. The first metal layer M1 may be deposited using an e-beam evaporator or a sputtering system. In example embodiments, the first metal layer M1 may include at least one of, for example, nickel, platinum, palladium, cobalt, rhodium, silver, gold, copper, iridium, rhenium, or cerium. The first metal layer M1 may be deposited to a thickness of several nanometers to several hundred nanometers (for example, 300 nm-1000 nm).

Referring to FIG. 7C, the second metal layer M2 may be formed on the first metal layer M1. The second metal layer M2 may be deposited using an e-beam evaporator or a sputtering system. The second metal layer M2 may include at least one of, for example, nickel, platinum, palladium, cobalt, rhodium, silver, gold, copper, iridium, rhenium, or cerium, and be selected to be different from the first metal layer M1. The second metal layer M2 may be deposited to a thickness of several nanometers to several hundred nanometers (for example, 300 nm-1000 nm). Accordingly, a total thickness of the first and second metal layers M1 and M2 may be about 1 μm to 2 μm on the semiconductor layer 200.

Referring to FIG. 7D, the photoresist pattern 250 and the first and second metal layers M1 and M2 on the photoresist pattern 250 may be removed, after the formation of the nano-metal rods NM on the semiconductor layer 200.

According to example embodiments of the inventive concept, a propulsive force of a nano-motor may vary depending on a change in concentration of target molecules, and the propulsive force of the nano-motor may be measured in a form of an electric signal. As a result, it is possible to realize a biosensor with a reduced size and a real-time detecting function.

According to example embodiments of the inventive concept, a nano-motor array may be formed on a silicon channel spaced or floated apart from a substrate. As the result of the floated disposition of the silicon channel, a stress applied to the silicon channel by the nano-motor array can be converted into the electrical signal, whose variation can be easily measured.

While example embodiments of the inventive concepts have been particularly shown and described, it will be understood by one of ordinary skill in the art that variations in form and detail may be made therein without departing from the spirit and scope of the attached claims.

What is claimed is:

1. A biosensor, comprising:
a supporting substrate;
a semiconductor layer spaced apart from a top surface of the supporting substrate by supporting patterns; and
a nano-motor array formed on a top surface of the semiconductor layer, the nano-motor array including a plurality of nano-metal rods configured to exhibit an autonomous propulsion in a fluid.

2. The biosensor of claim 1, wherein the nano-metal rod has a structure, in which different metal layers are jointed with each other.

3. The biosensor of claim 1, wherein the nano-metal rod is provided as a form of a Pt—Au metal pair or a Ni—Au metal pair.

4. The biosensor of claim 1, wherein the nano-metal rod is configured to be moved along a specific direction in a hydrogen peroxide solution, thereby exhibiting the autonomous propulsion.

5. The biosensor of claim 1, wherein the nano-metal rod is configured to apply a stress to the semiconductor layer, when the nano-motor array is provided in the fluid.

6. The biosensor of claim 1, wherein the semiconductor layer is formed of a semiconductor material, whose electric conductivity varies substantially depending on the autonomous propulsion from the nano-metal rods.

7. The biosensor of claim 1, wherein the semiconductor layer has a thickness ranging from 1 nm to 100 nm.

8. An apparatus for detecting a biomaterial, comprising:
a biomaterial reacting part immobilized with probe molecules, the probe molecules being specifically bound with target molecules labeled with silver nanoparticles;
a biomaterial detecting part provided with a nano-motor array including a plurality of nano-metal rods, the nano-metal rods exhibiting an autonomous propulsion in a fluid; and
a fluid channel supplying the fluid to the biomaterial reacting part and the biomaterial detecting part.

9. The apparatus of claim 8, wherein the biomaterial detecting part is configured to detect a change in electric conductivity of the semiconductor layer between before and after providing the nano-motor array in the fluid.

10. The apparatus of claim 8, wherein, in the biomaterial detecting part, the autonomous propulsion of the nano-metal rods is dependent on a concentration of silver ions dissolved in the fluid.

11. The apparatus of claim 8, wherein the biomaterial detecting part further comprises a supporting substrate, a semiconductor layer provided on a top surface of the supporting substrate by supporting patterns interposed between the supporting substrate and the semiconductor layer to separate the supporting substrate from the semiconductor layer, and
the nano-metal rods are provided on a top surface of the semiconductor layer.

12. The apparatus of claim 11, wherein the nano-metal rod is configured to apply a stress to the semiconductor layer, when the nano-motor array of the biomaterial detecting part is provided in the fluid.

13. The apparatus of claim 11, wherein the semiconductor layer is formed of a semiconductor material, whose electric conductivity varies substantially depending on the autonomous propulsion from the nano-metal rods.

14. The apparatus of claim 11, wherein each of the nano-metal rods is provided as a form of a Pt—Au metal pair or a Ni—Au metal pair configured to be moved along a specific direction in a hydrogen peroxide solution.

15. A method of detecting a biomaterial using a biomaterial-detecting apparatus with a biomaterial reacting part and a biomaterial detecting part that is formed on a semiconductor layer and is provided with a nano-motor array including a plurality of nano-metal rods, the nano-metal rods exhibiting an autonomous propulsion in a fluid,
wherein the method comprises:
measuring a first electric conductivity of the semiconductor layer;
immobilizing target molecules labeled with silver nanoparticles on the biomaterial reacting part;
supplying a fluid to the biomaterial reacting part immobilized with the target molecules and the biomaterial detecting part; and
measuring a second electric conductivity of the semiconductor layer, during the supplying of the fluid.

16. The method of claim 15, further comprising, determining a concentration of the target molecule using a difference between the first electric conductivity and the second electric conductivity.

17. The method of claim 16, wherein the determining of a concentration of the target molecule is performed by using a change in a concentration of silver ion caused by dissolution of the silver nanoparticle by the fluid.

18. The method of claim 15, wherein the nano-metal rods are provided as a form of a Pt—Au metal pair or a Ni—Au metal pair, and
the supplying of the fluid comprises supplying a hydrogen peroxide solution.

19. The method of claim 15, wherein the supplying of the fluid comprises supplying a hydrogen peroxide solution.

20. The method of claim 15, wherein the immobilizing of the target molecules labeled with silver nanoparticles comprises:
providing a substrate immobilized with first probe molecules to the biomaterial reacting part, the first probe molecule specifically bound with the target molecules;
providing the target molecules to the biomaterial reacting part to bind the target molecules specifically with the first probe molecules; and
providing second probe molecules immobilized on a surface of the silver nanoparticle to the biomaterial reacting part to bind the second probe molecules specifically with the target molecules.
